(19) United States
(12) Patent Application Publication
(10) Pub. No.: US 2026/0083370 A1
(43) Pub. Date: Mar. 26, 2026

(54) MONITORING, TRACKING, AND REPORTING USER EMOTIONAL STATES FROM A MANAGED DEVICE

(71) Applicant: MOTOROLA MOBILITY LLC, WILMINGTON, DE (US)

(72) Inventors: Amit Kumar Agrawal, Bangalore (IN); Jyothsna Bandameedipalli, Bangalore (IN); Thirumarai Selvi Seerangan, Bangalore (IN)

(21) Appl. No.: 18/896,627

(22) Filed: Sep. 25, 2024

(51) Int. Cl. A61B 5/16; A61B 5/0205; G06F 40/30; G08B 21/18

(52) U.S. Cl. CPC A61B 5/165; A61B 5/0205; G06F 40/30; G08B 21/182

(57) ABSTRACT

A method provides techniques for monitoring, tracking, and reporting user emotional states from a managed device. Data relating to communications and operations occurring on a first electronic device associated with a first user is monitored and recorded. Patterns associated with one or more emotional states of the first user are determined based on the monitored data. A report indicating dominant emotional states is generated and transmitted to a second electronic device associated with a second user. The second electronic device can be a managing device, where the managing device can monitor and/or control functionality of the first electronic device. Disclosed embodiments can include displaying an alert on a managing device when a negative emotional state or condition of a user associated with a managed device is detected.

### BACKGROUND

### 1. Technical Field

The present disclosure generally relates to electronic devices, and more specifically to electronic devices that support cross-device management and monitoring of usage of applications.

### 2. Description of the Related Art

Emotions are complex psychological states that involve a variety of feelings, thoughts, and behaviors. People experience a wide range of emotions on a frequent basis, often in response to different situations, interactions, or internal thoughts. Emotions can be positive, negative, or neutral. A positive emotion is characterized by feelings of contentment, joy, pleasure, and satisfaction. For example, happiness often arises from pleasant experiences, achievements, or social connections. A negative emotion can include one that involves feelings of loss, disappointment, grief, or helplessness. For example, sadness can be triggered by personal setbacks, the loss of a loved one, or witnessing suffering. Emotions can represent a broad spectrum of the human emotional experience. While some emotions (e.g., happiness, love, and gratitude) are positive and contribute to well-being, others (such as sadness, anger, and fear) are more challenging but also play essential roles in self-awareness, personal growth, and survival. Understanding and recognizing these emotions can help individuals manage them effectively and respond appropriately to different life situations. Oftentimes, the emotional state of an individual can be important for others to know about, particularly when the individual's emotional state is not positive.

### BRIEF DESCRIPTION OF THE DRAWINGS

The description of the illustrative embodiments can be read in conjunction with the accompanying figures. It will be appreciated that for simplicity and clarity of illustration, elements illustrated in the figures have not necessarily been drawn to scale. For example, the dimensions of some of the elements are exaggerated relative to other elements. Embodiments incorporating teachings of the present disclosure are shown and described with respect to the figures presented herein, in which:

FIG. 1 depicts an example component makeup of an electronic device with specific components that enable the device to implement monitoring, tracking, and reporting user emotional states from a managed device, according to one or more embodiments;

FIG. 2 is an example messaging conversation that can contribute to or influence or be correlated to a person's emotional state, according to one or more embodiments;

FIG. 3 is an exemplary operations record generated on a managed device to present data associated with/supporting a determined emotional state, according to one or more embodiments;

FIG. 4 is an example of a vulnerable state alert message presented on a display of a managing device, according to one or more embodiments;

FIG. 5 is an example of a sentiment report that can be presented on a managing device, according to one or more embodiments;

FIG. 6 is an example configuration user interface for a managing device to configure a managed device for emotional state monitoring and reporting, according to one or more embodiments;

FIG. 7 is an example of an assistance query presented on a managed device, according to one or more embodiments;

FIG. 8 depicts a flowchart of a computer-implemented method for monitoring, tracking, and reporting user emotional states from a managed device, according to one or more embodiments; and

FIG. 9 depicts a flowchart of a computer-implemented method for generating a report indicating emotional states based on collected data related to communications and operations, according to one or more embodiments.

### DETAILED DESCRIPTION

According to aspects of the present disclosure, an electronic device, a method, and a computer program product provide techniques for monitoring, tracking, and reporting user emotional states from a managed device. Data relating to communications and operations occurring on a first electronic device associated with a first user are monitored and recorded. Patterns associated with one or more emotional states of the first user are determined based on the monitored data. A report indicating dominant emotional states is generated and transmitted to a second electronic device associated with a second user. The second electronic device can be a managing device, where the managing device can monitor and/or control functionality of the first electronic device. The first electronic device can be a managed electronic device. In embodiments, the first user can be a child, and the second user can be a parent of the child. Accordingly, disclosed embodiments can include displaying an alert on a managing device when a negative emotional state or condition of a user associated with a managed device is detected.

The digital age has brought numerous advantages, including easy access to information, instant communication, and social connectivity. However, these benefits come with significant risks, especially for children and teenagers who may be more vulnerable to the darker sides of the internet. Social media, harmful websites, and cyber-bullying (e.g., through text messages) can pose serious dangers to young people, impacting their psychological well-being and, in extreme cases, leading to suicidal thoughts or actions.

Social media platforms can be breeding grounds for cyberbullying, where individuals use online channels to harass, threaten, or humiliate others. The anonymity and reach of these platforms can amplify the effects, making it hard for victims to escape their tormentors. Constant exposure to hurtful comments, rumors, or even doctored images can lead to anxiety, depression, and a sense of helplessness. Social media often showcases an idealized version of reality, leading young users to compare themselves unfavorably with others. Constant exposure to curated images of peers'seemingly perfect lives can lead to feelings of inadequacy, low self-esteem, and body image issues. This comparison culture can erode self-worth, making young people more susceptible to depression.

Harmful websites that promote violence, self-harm, eating disorders, or drug use can negatively influence young minds. Exposure to such content can normalize dangerous behaviors, making them seem acceptable or even desirable. Websites that spread false information or extremist ideologies can mislead young people, shaping their beliefs and attitudes in harmful ways. Vulnerable children might be influenced to adopt radical views or engage in risky behaviors based on misleading or false information.

Cyber-bullying through text messages can be relentless, as perpetrators can reach their victims anytime and anywhere. This constant exposure can make it feel like there is no safe space, leading to chronic stress, anxiety, and a sense of being trapped. Bullying through group texts or social media can quickly escalate, involving more participants and leading to public shaming or humiliation. The public nature of these attacks can be devastating, causing deep psychological scars. In person bullying can cause emotional/psychological harm as well, which can lead to on-device activity that is not wholesome or helpful. Constant exposure to cyber-bullying, in-person bullying, harmful content, and social media pressure can lead to chronic anxiety and depression. The feeling of being overwhelmed or constantly under attack can create a sense of hopelessness and despair. In severe cases, the psychological impact of cyber-bullying and exposure to harmful content can lead young people to engage in self-harm as a way to cope with their pain. Tragically, some may feel that suicide is the only way to escape their suffering, especially if they believe there is no one to turn to for support. Even if the immediate threat of cyber-bullying or exposure to harmful content ends, the emotional scars can last for years. These experiences can impact self-esteem, trust in others, and the ability to form healthy relationships. The affected individuals often turn inward and chose to no share their experiences with others, such as their parents, who care about them. Those individuals can be oblivious to the trauma being inflicted on or felt by their child, relative, friend, and, as a result, may not provide any support. Even grown-ups can be susceptible to emotional mode swings based on content consumption (and/or exposure) on their devices and in their daily life experiences.

The disclosed embodiments simplify the challenges of monitoring data relating to communications and operations on a managed device that is communicatively interconnected with a managing device. Disclosed embodiments can monitor communications and operations on a managed device, such as messages and messaging conversations, voice conversations, video conversations, media consumption, website activity, social media posts viewed and/or posted, and/or other operations and activities. An emotional state of the device user is inferred based on the data relating to communications and operations. One or more embodiments can include: performing a sentiment analysis based on the collected data; and determining a dominant emotion of the first user based on the sentiment analysis. A report indicating the inferred dominant emotional state is communicated to the managing device. In some embodiments, suggestions and/or warnings can be provided to the managing device in response to detecting an adverse emotional state on the managed device. In this way, for example, a parent can receive notifications if an adverse emotional state of his/her child is detected. Disclosed embodiments enable the managed device to perform one or more actions, including sending an alert to a managing device, locking down or disabling one or more features of the managed device, sending an assistance query to the managed device to ask if the user needs help, and/or other actions. Thus, disclosed embodiments provide techniques for an early intervention when a potentially unsafe emotional state is detected, thereby reducing the risk of adverse outcomes where someone hurts others and/or themselves while in an unsafe emotional state such as excessive depression, anger, or the like.

One or more embodiments can include an electronic device including a communications subsystem enabling the electronic device to communicatively connect to at least one second electronic device, wherein the electronic device is a managed electronic device and wherein the at least one second electronic device is a managing electronic device. The electronic device includes a memory having stored thereon an emotional state monitoring, tracking, and reporting (ESMTR) module that autonomously records data associated with an emotional state of a first user of the electronic device. The electronic device further includes at least one processor coupled to the communications subsystem and the memory and which processes program code of the ESMTR module, the at least one processor configured to cause the electronic device to: monitor and record data relating to communications and operations occurring on the electronic device of the first user; determine patterns associated with one or more emotional states of the first user based on the monitored data; generate a report indicating the one or more emotional states of the first user based on the determined patterns; and transmit, to a managing device of a second user, the generated report indicating the one or more emotional states of the first user of the managed electronic device.

Embodiments can provide a method that includes: monitoring, by one or more processors of a managed device of a first user, data relating to communications and operations occurring on the managed device of the first user; determining patterns associated with one or more emotional states of the first user based on the monitored data; generating a report indicating the one or more emotional states of the first user based on the patterns; and transmitting, to a managing device of a second user, the generated report indicating the one or more emotional states of the first user of the managed device.

Further embodiments can provide a computer program product including: a non-transitory computer readable medium; and program code on the computer readable medium that when processed by a processor of an electronic device configures the processor to perform functions of the above-described method.

The above descriptions contain simplifications, generalizations and omissions of detail and is not intended as a comprehensive description of the claimed subject matter but, rather, is intended to provide a brief overview of some of the functionality associated therewith. Other systems, methods, functionality, features, and advantages of the claimed subject matter will be or will become apparent to one with skill in the art upon examination of the figures and the remaining detailed written description. The above as well as additional objectives, features, and advantages of the present disclosure will become apparent in the following detailed description.

Each of the above and below described features and functions of the various different aspects, which are presented as operations performed by the processor(s) of the communication/electronic devices are also described as features and functions provided by a plurality of corresponding methods and computer program products, within the various different embodiments presented herein. In the embodiments presented as computer program products, the computer program product includes a non-transitory computer readable storage device having program instructions or code stored thereon, and configuring the electronic device and/or host electronic device to complete the functionality of a respective one of the above-described processes when the program instructions or code are processed by at least one processor of the corresponding electronic/communication device, such as is described above.

In the following description, specific example embodiments in which the disclosure may be practiced are described in sufficient detail to enable those skilled in the art to practice the disclosed embodiments. For example, specific details such as specific method orders, structures, elements, and connections have been presented herein. However, it is to be understood that the specific details presented need not be utilized to practice embodiments of the present disclosure. It is also to be understood that other embodiments may be utilized and that logical, architectural, programmatic, mechanical, electrical and other changes may be made without departing from the general scope of the disclosure. The following detailed description is, therefore, not to be taken in a limiting sense, and the scope of the present disclosure is defined by the appended claims and equivalents thereof.

References within the specification to “one embodiment,” “an embodiment,” “embodiments”, or “one or more embodiments” are intended to indicate that a particular feature, structure, or characteristic described in connection with the embodiment is included in at least one implementation (embodiment) of the present disclosure. The appearance of such phrases in various places within the specification are not necessarily all referring to the same embodiment, nor are separate or alternative embodiments mutually exclusive of other embodiments. Further, various features are described which may be exhibited by some embodiments and not by others. Similarly, various aspects are described which may be aspects for some embodiments but not for other embodiments.

The terminology used herein is for the purpose of describing particular embodiments only and is not intended to be limiting of the disclosure. As used herein, the singular forms “a”, “an”, and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Moreover, the use of the terms first, second, etc. do not denote any order or importance, but rather the terms first, second, etc. are used to distinguish one element (e.g., a person or a device) from another.

It is understood that the use of specific component, device and/or parameter names and/or corresponding acronyms thereof, such as those of the executing utility, logic, and/or firmware described herein, are for example only and not meant to imply any limitations on the described embodiments. The embodiments may thus be described with different nomenclature and/or terminology utilized to describe the components, devices, parameters, methods and/or functions herein, without limitation. References to any specific protocol or proprietary name in describing one or more elements, features or concepts of the embodiments are provided solely as examples of one implementation, and such references do not limit the extension of the claimed embodiments to embodiments in which different element, feature, protocol, or concept names are utilized. Thus, each term utilized herein is to be provided its broadest interpretation given the context in which that term is utilized.

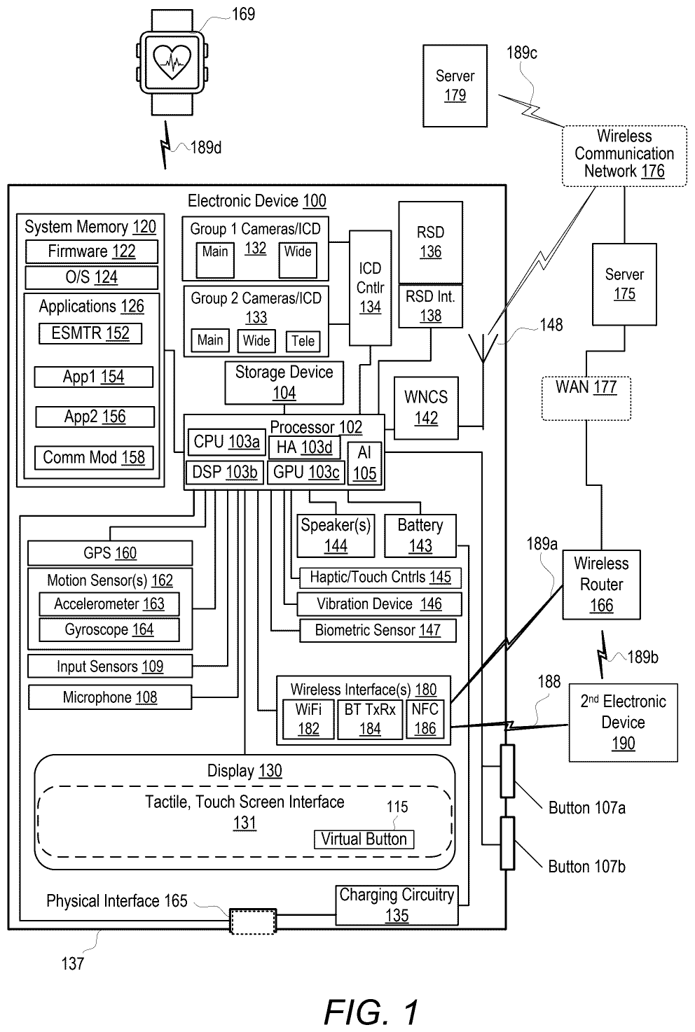

Those of ordinary skill in the art will appreciate that the hardware components and basic configuration depicted in the following figures may vary. For example, the illustrative components within electronic device 100 (FIG. 1) are not intended to be exhaustive, but rather are representative to highlight components that can be utilized to implement the present disclosure. For example, other devices/components may be used in addition to, or in place of, the hardware depicted. The depicted example is not meant to imply architectural or other limitations with respect to the presently described embodiments and/or the general disclosure. Throughout this disclosure, the terms ‘electronic device’, ‘communication device’, and ‘electronic communication device’ may be used interchangeably, and may refer to devices such as smartphones, tablet computers, and/or other computing/communication devices.

Within the descriptions of the different views of the figures, the use of the same reference numerals and/or symbols in different drawings indicates similar or identical items, and similar elements can be provided similar names and reference numerals throughout the figure(s). The specific identifiers/names and reference numerals assigned to the elements are provided solely to aid in the description and are not meant to imply any limitations (structural or functional or otherwise) on the described embodiments.

Referring now to the figures and beginning with FIG. 1, there is illustrated an example component makeup of electronic device 100, within which various aspects of the disclosure can be implemented, according to one or more embodiments. Electronic device 100 includes specific components that configure the device for monitoring, tracking, and reporting user emotional states from a managed device, according to one or more embodiments. Examples of electronic device 100 include, but are not limited to, mobile devices, a notebook computer, a mobile phone, a smart phone, a digital camera with enhanced processing capabilities, a smart watch, a tablet computer, and other types of electronic device.

Electronic device 100 includes processor 102 (typically as a part of a processor integrated circuit (IC) chip), which includes processor resources such as central processing unit (CPU) 103a, communication signal processing resources such as digital signal processor (DSP) 103b, graphics processing unit (GPU) 103c, and hardware acceleration (HA) unit 103d. In some embodiments, the hardware acceleration (HA) unit 103d may establish direct memory access (DMA) sessions to route network traffic to various elements within electronic device 100 without direct involvement from processor 102 and/or operating system 124. Processor 102 can interchangeably be referred to as controller 102.

Processor 102 can, in some embodiments, include image signal processors (ISPs) (not shown) and dedicated artificial intelligence (AI) engines 105. In one or more embodiments, processor 102 can execute AI modules to provide AI functionality of AI engines 105. AI modules may include an artificial neural network, a decision tree, a support vector machine, Hidden Markov model, linear regression, logistic regression, Bayesian networks, and so forth. The AI modules can be individually trained to perform specific tasks and can be arranged in different sets of AI modules to generate different types of output. Processor 102 is communicatively coupled to storage device 104, system memory 120, input devices (introduced below), output devices, including integrated display 130, and image capture device (ICD) controller 134.

For simplicity in describing the features of the electronic device 100, the functionality provided by one or more of CPU 103a, DSP 103b, GPU 103c, and ICD controller 134 are collectively described as being performed by processor 102. Collectively, components integrated within processor 102 support computing, classifying, processing, transmitting and receiving of data and information, and presenting of graphical images within a display.

System memory 120 may be a combination of volatile and non-volatile memory, such as random-access memory (RAM) and read-only memory (ROM). System memory 120 can store program code or similar data associated with firmware 122, an operating system 124, and/or applications 126. During device operation, processor 102 processes program code of the various applications, modules, OS, and firmware, that are stored in system memory 120.

In accordance with one or more embodiments, applications 126 include, without limitation, emotional state monitoring, tracking, and reporting (ESMTR) module 152, other applications, indicated as App1 154 and App2 156, and communication module 158. Other applications may also be present. Each module and/or application provides program instructions/code that are processed by processor 102 to cause processor 102 and/or other components of electronic device 100 to perform specific operations, as described herein. Descriptive names assigned to these modules add no functionality and are provided solely to identify the underlying features performed by processing the different modules. For example, emotional state monitoring, tracking, and reporting (ESMTR) module 152 can include program instructions for implementing features of the disclosed embodiments. The ESMTR module 152 can include instructions that cause or configure processor 102 to monitor and record data relating to communications, operations, and/or other user activity. Based on the data, a dominant emotional state of a user is determined, along with a level of the emotion. In response to determining an elevated level of a potentially dangerous emotion (e.g., depression, anger, panic, etc.), one or more mitigation actions can be taken. These actions can include, but are not limited to, generating a report indicating the emotional states of the user of a managed electronic device, and providing the report to a managing electronic device. Other features are described in further detail throughout this disclosure.

ICD controller 134 can perform image acquisition functions in response to commands received from processor 102 in order to control group 1 ICDs 132 and group 2 ICDs 133 to capture video or still images of a local scene within a FOV of the operating/active ICD. In one or more embodiments, group 1 ICDs can be front-facing, and group 2 ICDs can be rear-facing, or vice versa. Throughout the disclosure, the term image capturing device (ICD) is utilized interchangeably to be synonymous with and/or refer to any one of the cameras 132, 133. Both sets of cameras 132, 133 include image sensors that can capture images that are within the field of view (FOV) of the respective camera 132, 133.

In one or more embodiments, the functionality of ICD controller 134 is incorporated within processor 102, eliminating the need for a separate ICD controller. Thus, for simplicity in describing the features presented herein, the various camera selection, activation, and configuration functions performed by the ICD controller 134 are described as being provided generally by processor 102. Similarly, manipulation of captured images and videos are typically performed by GPU 103c, and certain aspects of device communication via wireless networks are performed by DSP 103b, with support from CPU 103a. In one or more embodiments, electronic device 100 includes removable storage device (RSD) 136, which is inserted into RSD interface 138 that is communicatively coupled via system interlink to processor 102. In one or more embodiments, RSD 136 is a non-transitory computer program product or computer readable storage device encoded with program code and corresponding data, and RSD 136 can be interchangeably referred to as a non-transitory computer program product. RSD 136 may have a version of one or more applications stored thereon. Processor 102 can access RSD 136 to provision electronic device 100 with program code that, when executed/processed by processor 102, the program code causes or configures processor 102 and/or generally electronic device 100, to provide the various functions described herein.

Electronic device 100 includes an integrated display 130 which incorporates a tactile, touch screen interface 131 that can receive user tactile/touch input. As a touch screen device, integrated display 130 allows a user to provide input to or to control electronic device 100 by touching features within the user interface presented on display 130. Tactile, touch screen interface 131 can be utilized as an input device. The touch screen interface 131 can include one or more virtual buttons, indicated generally as 115. In one or more embodiments, when a user applies a finger or stylus on the touch screen interface 131 in the region demarked by the virtual button 115, the touch of the region causes the processor 102 to execute code to implement a function associated with the virtual button. In some implementations, integrated display 130 is integrated into a front surface of electronic device 100 along with front ICDs, while the higher quality ICDs are located on a rear surface. Other embodiments provide for multiple integrated displays within electronic device 100 and references to integrated display 130 are assumed to refer to one or all of these multiple integrated displays.

Electronic device 100 can further include microphone 108, one or more output devices such as speakers 144, and one or more input buttons, indicated as 107a and 107b. While two buttons are shown in FIG. 1, other embodiments may have more or fewer input buttons. Microphone 108 can also be referred to as an audio input device. In some embodiments, microphone 108 may be used for identifying a user via voiceprint, voice recognition, and/or other suitable techniques. Input buttons 107a and 107b may provide controls for volume, power, and ICDs 132, 133. Additionally, electronic device 100 can include input sensors 109 (e.g., sensors enabling gesture detection by a user).

Electronic device 100 further includes haptic touch controls 145, vibration device 146, fingerprint/biometric sensor 147, global positioning system (GPS) module 160, and motion sensor(s) 162. Vibration device 146 can cause electronic device 100 to vibrate or shake when activated. Vibration device 146 can be activated during an incoming call or message in order to provide an alert or notification to a user of electronic device 100. In one or more embodiments, integrated display 130, speakers 144, and vibration device 146 can generally and collectively be referred to as output devices.

Biometric sensor 147 can be used to read/receive biometric data, such as fingerprints, to identify or authenticate a user. In some embodiments, the biometric sensor 147 can supplement an ICD (camera), which provides facial recognition for user detection/identification.

GPS module 160 can provide time data and location data about the physical location of electronic device 100 using geospatial input received from GPS satellites. Motion sensor(s) 162 can include one or more accelerometers 163 and gyroscope 164. Motion sensor(s) 162 can detect movement of electronic device 100 and provide motion data to processor 102 indicating the spatial orientation and movement of electronic device 100. Accelerometers 163 measure linear acceleration of movement of electronic device 100 in multiple axes (X, Y and Z). Gyroscope 164 measures rotation or angular rotational velocity of electronic device 100. Electronic device 100 further includes a housing 137 (generally represented by the thick exterior rectangle) that contains/protects the components internal to electronic device 100.

Electronic device 100 also includes a physical interface 165. Physical interface 165 of electronic device 100 can serve as a data port and can be used as a power supply port that is coupled to charging circuitry 135 and device battery 143 to enable recharging of device battery 143 and/or powering of device.

Electronic device 100 further includes wireless network communication subsystem (WNCS) 142, which can represent one or more front end devices (not shown) that are each coupled to one or more antennas 148. In one or more embodiments, WNCS 142 can include a communication module with one or more baseband processors or digital signal processors, one or more modems, and a radio frequency (RF) front end having one or more transmitters and one or more receivers. Example communication module 158 within system memory 120 enables electronic device 100 to communicate with wireless communication network 176 and with other devices, such as server 175 and other connected devices, such as second electronic device 190. via one or more of data, audio, text, and video communications. Communication module 158 can support various communication sessions by electronic device 100, such as audio communication sessions, video communication sessions, text communication sessions, exchange of data, and/or a combined audio/text/video/data communication session.

WNCS 142 and antennas 148 allow electronic device 100 to communicate wirelessly with wireless communication network 176 via transmissions of communication signals to and from network communication devices, such as base stations or cellular nodes, of wireless communication network 176. Wireless communication network 176 further allows electronic device 100 to wirelessly communicate with server 175, and other communication devices, such as second electronic device 190, which can be similarly connected to wireless communication network 176. In one or more embodiments, various functions that are being performed on communications device 100 can be supported using or completed via/on server 175. Second electronic device 190 can be a communication device, such as a smartphone. In one or more embodiments, a wearable computing device 169, such as a smartwatch, fitness tracker, or the like, may be paired with electronic device 100, and provide biometric data such as heart rate, to the electronic device 100.

In one or more embodiments, electronic device 100 can communicate wirelessly with external wireless devices, such as a WiFi router 166 or second electronic device 190, via one or more of short-range wireless interface(s) 180. In one or more embodiments, WiFi router 166 may be connected to a WAN 177 and/or server 175. Electronic device 100 can wirelessly communicate with second electronic device 190 via communication signal 188 (communicating between wireless interface(s) 180 and second electronic device 190). Communication signal 189a and communication signal 189b provide a communication path from electronic device 100 to second electronic device 190. In one or more embodiments, signals 188 and/or signals 189a may be transmitted by short range communication device(s) within wireless interface(s) 180. Server 175 may also be connected to wireless communication network 176, which may enable connection to other servers, such as server 179 via communication signal 189c. Communication signal 189d may be used to enable communication between electronic device 100 and wearable computing device 169. Wireless interface(s) 180 can include short-range wireless communication adapters/transceivers, such as wireless fidelity (Wi-Fi) transceiver 182 for Wi-Fi connectivity, Bluetooth transceiver 184, and near field communication (NFC) transceiver 186. In one or more embodiments, electronic device 100 can receive Internet or Wi-Fi based calls, text messages, multimedia messages, and other notifications via wireless interface(s) 180. In one or more embodiments, WNCS 142 with antenna(s) 148 and wireless interface(s) 180 collectively provide/represent the wireless communications subsystem of electronic device 100. Electronic device 100 of FIG. 1 is only a specific example of a device that can be used to implement the embodiments of the present disclosure.

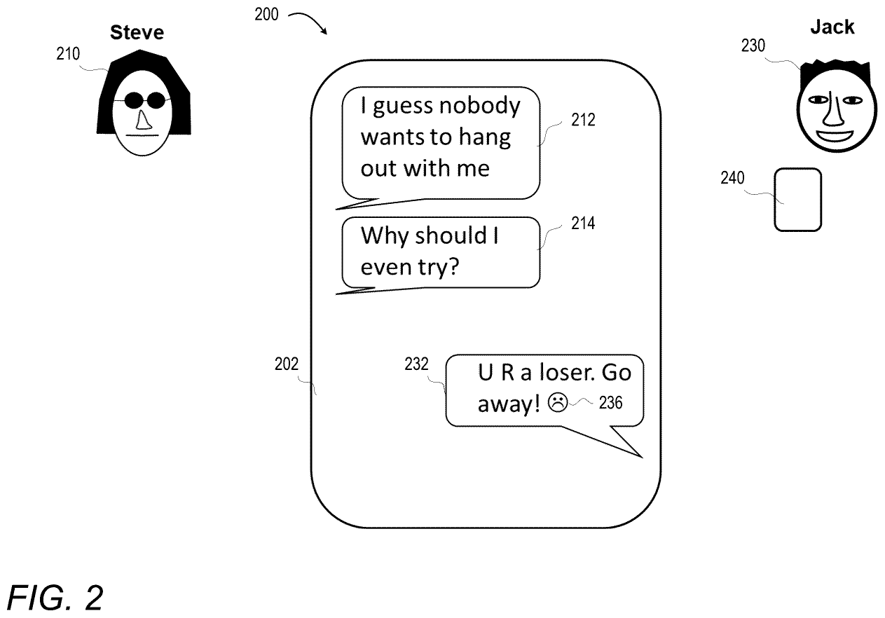

FIG. 2 is an example messaging exchange occurring on a managed device involving the device user and a second person, who has a separate device. Device 200 and device 240 may be similar to device 100 described in FIG. 1. In the example shown in FIG. 2, device 200 is associated with (belongs to) user 210, indicated as ‘Steve.’ The display 202 of device 200 shows a conversation between user 210 and a second device user 230, indicated as ‘Jack.’ Message 212 and message 214 originate from device 200 (i.e., entered by second device user 210), and message 232 originates from remote device 240 which is associated with user 230.

In one or more embodiments, the ESMTR module 152 (FIG. 1) configures electronic device 200 to monitor and record data relating to communications and operations occurring on the electronic device 200. In one or more embodiments, the monitoring and recording of data includes monitoring incoming and outgoing network traffic to obtain outgoing and incoming communications data and/or operations data. In one or more embodiments, the monitoring and recording of data includes performing optical character recognition (OCR) on image data presented on display 202 to obtain text from the image data. The text data can include ASCII text data, Unicode text data, and/or other suitable text representation. The text data can further include emoji data that represents an emoji, such as indicated at 236. The ESMTR module 152 (FIG. 1) may further perform and/or obtain sentiment analysis of the text and/or emoji data. As an example, message 212 states “I guess nobody wants to hang out with me.” In a similar theme, message 214 states “Why should I even try?” In some embodiments, the text from messages 212 and 214 are provided to a machine learning system that performs natural language processing (NLP) including sentiment analysis, subject detection, and/or other language analysis. Based on the content of the messages 212 and 214, the sentiment analysis may determine one or more dominant emotions that include sadness, discouragement, and resignation, pointing to a predominantly negative sentiment. In one or more embodiments, to monitor and record data relating to communications occurring on the electronic device of the first user, the at least one processor is further configured to obtain data from at least one outgoing message.

In a similar manner, the contents of incoming message 232 received at the device from the second device user, including the emoji 236, may be provided to the machine learning system that performs natural language processing (NLP) including sentiment analysis, subject detection, and/or other language analysis. Based on the content of incoming message 232, the sentiment analysis may determine a highly negative sentiment, where the incoming message 232 conveys disrespect and contempt, along with rejection. Thus, the incoming message 232, received from second device user 230, may be identified as hostile, dismissive, and unkind, reflecting a clear and strong negative sentiment. Disclosed embodiments may identify the sad or discouraged emotional state of user 210, and also identify a sentiment of the incoming message 232 as hostile, dismissive, and unkind, reflecting a clear and strong negative sentiment. In one or more embodiments, the combination of the sad emotional state, and the receipt of incoming messages with a strong negative sentiment can be used as criteria to determine a state of vulnerability and/or the potential for dangerous behaviors by the user 230. The dangerous behaviors could include inducing harm to one's self and/or to others. In response to determining a state of vulnerability and/or dangerous behavior potential, the disclosed embodiments can take one or more actions, including generating a report and sending the report to a managing device, displaying a message on the managed device, restricting capabilities of the managed device, and/or other suitable actions.

The message conversation shown in FIG. 2 is one example of data that may be recorded and monitored for implementing disclosed embodiments. However, there are various other types of activities for which data relating to communications and operations can be monitored and recorded. The activities can include reading social media content and/or reading website content. The activities can include posting social media content, posting to a blog, and/or other content creation activities. The activities can include media consumption. The media consumption can include listening to music on audio websites, such as Spotify, online radio stations, and/or other sources of music. The media consumption can include listening to podcasts. The media consumption can include watching videos on video sharing sites such as YouTube, and/or other sources of media. In one or more embodiments, the activities are recorded in an operations record. In one or more embodiments, the operations record may include a summary of activity over a predetermined time period (e.g., a 24-hour period). The operations record can include a list of applications (apps) used, content consumed, browsing history, and/or other suitable information that can be used in determining an emotional state of a user.

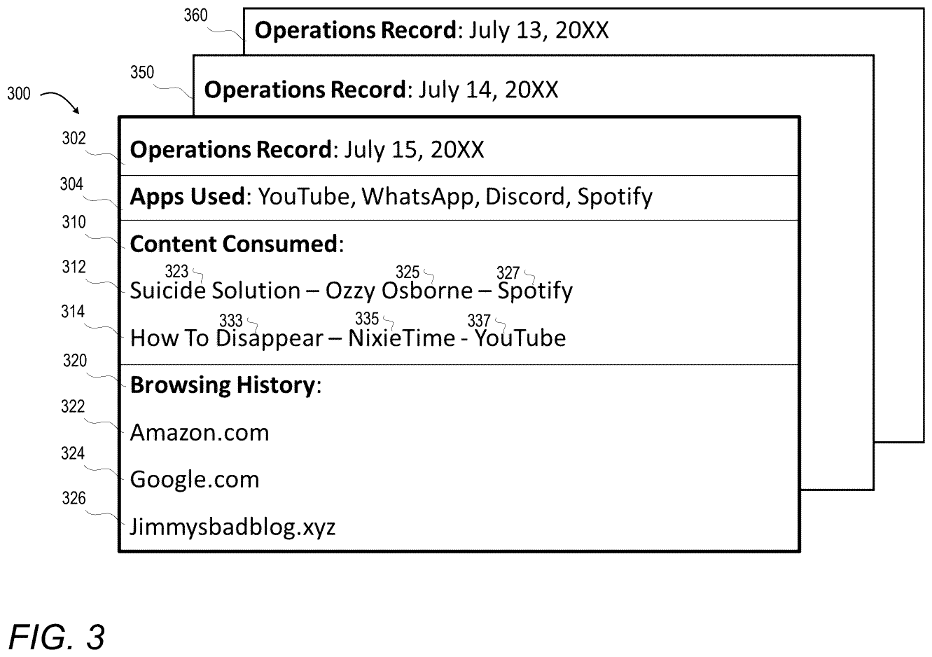

FIG. 3 is an exemplary operations record 300, according to one or more embodiments. Operations record 300 includes time interval 302. In one or more embodiments, the time interval can include a 24-hour time interval corresponding to a calendar date, a week interval, or an interval that is less than one day, such as a 4-hour interval, 6-hour interval, or other suitable time interval. The operations record 300 can include a list of applications used at 304. The operations record 300 can include a content consumption field 310. The content consumption field 310 can include one or more assets that have been at least partially consumed by user of the electronic device during the time interval 302. In the example of FIG. 3, there are two content records, indicated at 312 and 314. In one or more embodiments, the content can include a name, artist, and/or source of the content. In the example indicated at 312, the name 323 can be the name of a song, the artist 325 can be the artist that created the song, and the source 327 can be the website or service where the content was obtained. Similarly, in the example indicated at 314, the name 333 can be the title of a video, the source 335 can be the name of a channel on a video streaming site, and the source 337 can be the website or service where the content was obtained. The operations record 300 can include a browsing history field 320. The browsing history field 320 can include one or more websites that have been visited (accessed by the electronic device) during the time interval 302. In the example of FIG. 3, there are three entries, indicated as 322, 324, and 326. In one or more embodiments more than one operations record may be stored. As shown in FIG. 3, additional operations records, indicated as 350 and 360 are also stored. The additional operations records may cover different time intervals. In embodiments, the time intervals covered by stored operations records may be sequential. In the example of FIG. 3, operations record 350 pertains to a time interval covering July 14, and operations record 360 pertains to a time interval covering July 13. Accordingly, the operations records 300, 350, and 360 include a three-day interval of operational data, enabling the identification of trends and patterns. In one or more embodiments, the content indicated in content consumption field 310 may be analyzed for sentiment, to determine if the content is generally negative or generally positive. The determination may be made utilizing machine learning systems that can perform content analysis, including text analysis, natural language processing, image analysis, audio analysis, and/or other types of content analysis. In one or more embodiments, a sentiment trend is computed. If the sentiment trend is trending lower, meaning that the sentiment conveyed by the data relating to operations and communications is continuing to get more negative as time continues, then in response, one or more actions, including generating a vulnerable state alert message, and transmitting the vulnerable state alert message to a managing electronic device can be performed. In one or more embodiments, the multiple operations records 300, 350, and 360 can serve to determine a timeframe for which the one or more emotional states of the first (managed) user has been ongoing. In some embodiments, the operations records can be subject-based or emotion-based (rather than time based). With these embodiments, the content of the operations record can be amassed over a period of time to show more of a ramping-up towards the reported emotional state, based on a series/sequence of activity detected over the extended period of time. For example, a person can experience frequent depression, while not necessarily experiencing constant depression. Accordingly, disclosed embodiments can monitor and record a number of times within a given duration (e.g., a week, a month, etc.) that a depressed state is detected based on communications and/or operations data of a managed device, and issue a vulnerable state alert to a managing device in response to the number of times that depression is detected within the given duration exceeding a predetermined number of times.

One or more embodiments can include performing a sentiment analysis on the at least one outgoing message, and determining a sentiment score based on the sentiment analysis. In one or more embodiments, a sentiment score is computed, where a higher sentiment score equates to a more intense level of a negative emotional state, such as sad, depressed, angry, anxious, nervous, paranoid, and/or other negative emotional state. One or more embodiments can include: performing a sentiment analysis on the at least one outgoing message; and determining a sentiment score based on the sentiment analysis. One or more embodiments can include: in response to the sentiment score being above a predetermined threshold: generating a vulnerable state alert message; and transmitting the vulnerable state alert message to the at least one second electronic device. In one or more embodiments, the sentiment scores may be generated based on machine learning models such as Naive Bayes, Support Vector Machines (SVM), Random Forests, or neural networks on labeled datasets containing text and corresponding sentiment labels (e.g., happy, sad, angry). The models can be trained to classify sentiment based on features extracted from the training data. In embodiments, the models can be trained/configured to output probability scores for different sentiment classes. For example, a text might have an 80 percent probability of being classified as “sad” and a 20 percent probability of being “neutral.” One or more embodiments can include: obtaining data from at least one incoming message; performing a sentiment analysis on the at least one incoming message; and determining a sentiment score based on the sentiment analysis of the at least one outgoing message and the at least one incoming message.

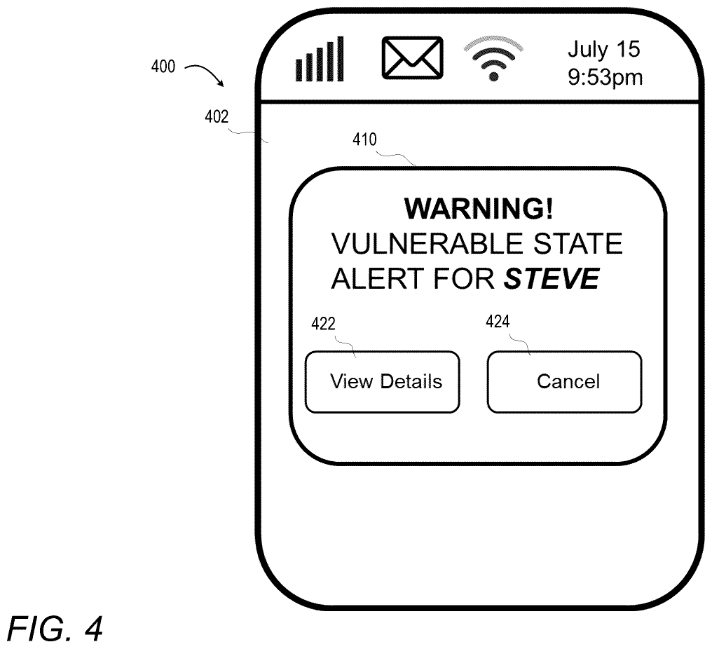

FIG. 4 is an example of a vulnerable state alert message, according to one or more embodiments. Device 400 may be configured similar to device 100 shown in FIG. 1. Device 400 includes an electronic display 402. Presented and rendered on display 402 is vulnerable state alert message 410. The vulnerable state alert message 410 may be rendered and presented on a managing device. In one or more embodiments, the second device (e.g., device 190 in FIG. 1) is a managing device associated with a managed device (e.g., device 100 in FIG. 1). The vulnerable state alert message 410 can include a cancel button 424. In response to detecting an invocation of the cancel button 424, the vulnerable state alert message 410 may be removed from the display 402. The vulnerable state alert message 410 can include a ‘view details’ button 422. In response to detecting an invocation of the ‘view details’ button 422, additional information can be shown on the display 402, such as a sentiment report that can show more details about what caused the vulnerable state alert to be generated and/or recommendations for the user of the managing device to consider.

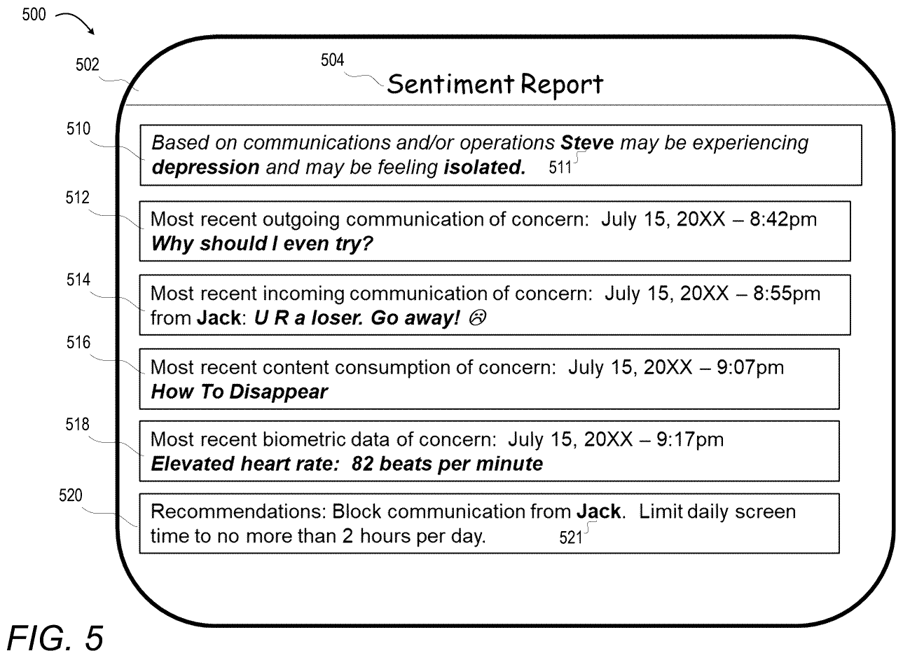

FIG. 5 is an example of a sentiment report presented on a display device of device 500, according to one or more embodiments. The sentiment report may be transmitted and/or otherwise provided to a managing device. The managing device can be associated with a caregiver of the person associated with the managed device. As an example, the managed device can be associated with a child, and the managing device can be associated with a parent of the child. Device 500 may be similar to device 100 shown in FIG. 1. Device 500 may be a managing device. Device 500 includes an electronic display 502. Presented and rendered on display 502 is sentiment report 504. The sentiment report 504 can include one or more fields. In some embodiments, the sentiment report 504 can include a sentiment summary field 510 that indicates one or more dominant emotions that are detected based on communications and/or operations on the managed electronic device. As an example, the sentiment summary field indicates a subject person 511, and one or more emotions, such as depression and feeling isolated. In embodiments, the sentiment report 504 can include an outgoing communication field 512 that indicates a most recent outgoing communication of concern, e.g., an outgoing communication having a negative sentiment. In one or more embodiments, the sentiment report 504 can include an incoming communication field 514 that indicates a most recent incoming communication of concern, which pertains to an incoming communication having a negative sentiment. In embodiments, the sentiment report 504 can include a content consumption field 516 that indicates a most recent content consumption of concern, which pertains to a consumed piece of content that is deemed to have a negative sentiment. In one or more embodiments, determining that a piece of consumed content has negative sentiment can be based on metadata associated with the content, a parental rating, analysis of spoken dialog, displayed text, and/or imagery within the content, and/or other criteria. The information displayed in fields 512, 514, and/or 516 can convey a root cause of, or suspected trigger for the one or more emotional states of the first (managed) user, and/or the monitored data that caused or triggered the one or more emotional states of the first user.

In some embodiments, the sentiment report 504 can include a biometric data field 518 that indicates a most recent biometric data measurement of concern. Elevated stress often leads to an increase in heart rate. The autonomic nervous system (ANS), particularly the sympathetic branch, triggers this response to prepare the body for a “fight-or-flight” reaction. Additionally, heart rate variability (HRV), which measures the variation in time between heartbeats, can correlate to levels of stress, where an increased HRV can indicate less adaptability of the cardiovascular system to changing conditions. Moreover, stress can cause both systolic and diastolic blood pressure to rise due to increased sympathetic nervous system activity. In one or more embodiments, the biometric data can include a heart rate, a heart rate variability measure, a blood pressure, a breathing rate, a body temperature, a perspiration level, and/or other suitable biometric measurements. In one or more embodiments, the biometric data may be obtained from a wearable device such as a smartwatch, fitness monitor, and/or other wearable device that is paired with the managed electronic device.

In some embodiments, the sentiment report 504 can include a recommendation field 520 that indicates one or more recommendations. One or more embodiments can include: generating, via an artificial intelligence engine, a recommendation indicating one or more remedial actions that can be taken by the second user of the managing device to address the one or more emotional states of the first user of the managed device; and transmitting the recommendation to the managing device along with the report. In some embodiments, the recommendations can be targeted to the managing user, and thus, are rendered on the managing electronic device. In embodiments, one or more recommendations can also be targeted to the managed user and thus, are rendered on the managed electronic device. In one or more embodiments, the recommendations can be based on the data relating to communications and/or operations of the managed device. In the example conversation shown in FIG. 2, there is an incoming message 232 that is determined to be hostile, dismissive, and unkind, reflecting a clear and strong negative sentiment. One or more embodiments can include recommending to block sources of negative sentiment. Accordingly, the recommendation in recommendation field 520 indicates that the sender of message 232 (user 230 ‘Jack’), indicated at 521, should be blocked. The blocking action prevents messages and/or incoming calls from the device 240 associated with user 230 (FIG. 2). In one or more embodiments, a block list is established, containing telephone numbers and/or other identifiers to determine a source of an incoming call or message that should be blocked. The at least one processor within the managed electronic device checks incoming text messages against the block list. If a match is found, the message is filtered out. In embodiments, blocked messages are not displayed to the user and may be silently discarded. In this way, the antagonizing incoming messages are blocked, which can help improve the emotional state of the recipient. Additionally, one or more embodiments can include recommendations to limit daily usage of the managed electronic device (i.e., ‘screen time’). Other recommendations are possible in disclosed embodiments. The other recommendations can include, but are not limited to, recommending blocking of a particular website, and/or recommending blocking of a particular content channel. Accordingly, disclosed embodiments can serve to alert parents if the emotional state of their child is declining, and/or if the child is being bullied online, and/or if the child consuming content that could exacerbate a negative emotional state. This awareness allows parents to intervene early, preventing emotional distress and harm. In one or more embodiments, the at least one processor is further configured to monitor at least one of: at least one browser search history of the first user of the electronic device; at least one application activity of the first user of the electronic device; and at least one content consumed by the first user of the electronic device. In one or more embodiments, the sentiment report provides at least one of: a description of the one or more emotional states of the first user; a timeframe for which the one or more emotional states of the first user has been ongoing; a root cause of, or suspected trigger for the one or more emotional states of the first user; and the monitored data that caused or triggered the one or more emotional states of the first user.

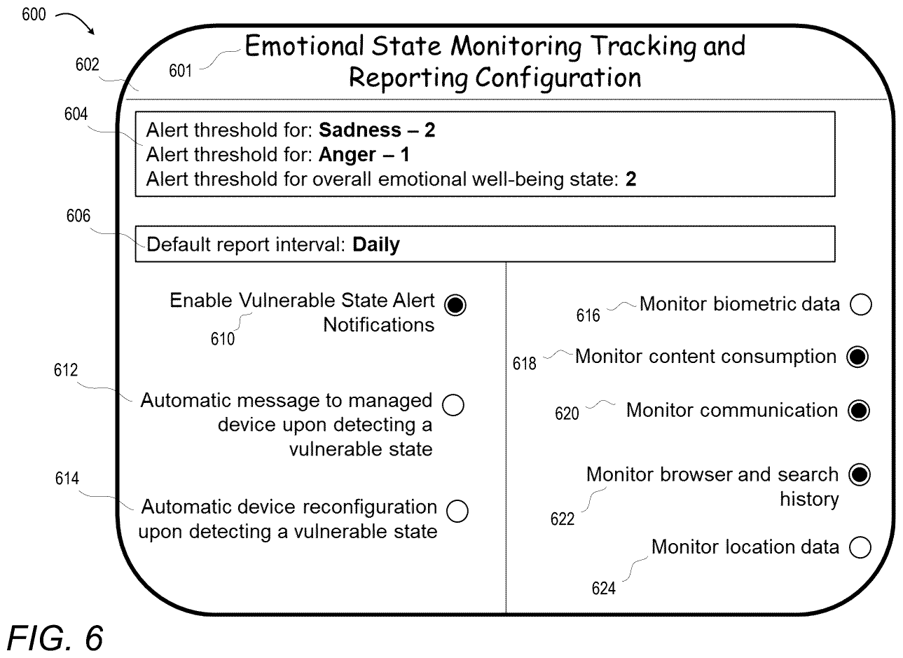

FIG. 6 is an example configuration user interface, according to one or more embodiments. Device 600 may be similar to device 100 shown in FIG. 1. Device 600 includes an electronic display 602. Presented and rendered on display 602 is emotional state monitoring, tracking, and reporting configuration user interface 601. In embodiments, the emotional state monitoring, tracking, and reporting (EMSTR) configuration user interface 601 can include an alert threshold field 604 that displays and allows setting of alert thresholds for one or more emotional states. The emotional states can include an overall emotional state, as well as levels of specific emotions such as sadness, anger, depression, anxiety, and the like. In one or more embodiments, the overall emotional state may be computed as an average of levels of multiple emotions, such as sadness, anger, depression, anxiety, and the like. In one or more embodiments, in response to determining a level of an emotion or an overall emotional well-being state that exceeds the specified threshold, an alert is generated and sent to the managing electronic device.

In embodiments, the EMSTR configuration user interface 601 can include a report interval 606. The report interval enables the managing user to specify how often a sentiment report is generated. In one or more embodiments, report interval options include hourly, daily, weekly, and monthly. Other report interval options are possible in disclosed embodiments. In embodiments, the EMSTR configuration user interface 601 can include an option 610 to enable vulnerable state alert notifications. With option 610 enabled, a managing device receives an alert when a vulnerable state is detected. The vulnerable state can be determined based on data relating to communications and/or operations of the managed electronic device.

In one or more embodiments, the EMSTR configuration user interface 601 can include an option 612 to enable an automatic message to be sent to the managing electronic device upon detecting a vulnerable state. With option 612 enabled, a managed device receives an alert when a vulnerable state is detected. Option 612 provides direct contact to the managed user, which can be the fastest way to reach the managed user, since the direct contact does not require any actions by the managing user.

In one or more embodiments, the EMSTR configuration user interface 601 can include an option 614 to enable automatic reconfiguration of the managed electronic device upon detecting a vulnerable state. With option 614 enabled, a managed device is reconfigured when a vulnerable state is detected. The reconfiguring can include restricting one or more features/functions of the managed electronic device. The restricting can include blocking usage of an application, blocking access to a website, blocking incoming calls and/or messages from a particular device/account/user, and so on.

In one or more embodiments, the EMSTR configuration user interface 601 can include an option 616 to enable monitoring of biometric data. With option 616 enabled, biometric data, such as heart rate, heart rate variability, breathing rate, body temperature, galvanic skin response (GSR), blood pressure, and/or other suitable biometric data is monitored. In one or more embodiments, the biometric data is used as criteria in determining an emotional state and/or a level of intensity of a particular emotion. In one or more embodiments, the biometric data may be acquired via a wearable electronic device such as a smartwatch or fitness tracker that is paired to the managed electronic device. In one or more embodiments, to determine patterns associated with one or more emotional states of the first user based on the monitored data, the at least one processor is further configured to monitor biometric data from among a group comprising at least one of heart rate, electrodermal activity, and respiratory pattern.

In one or more embodiments, the EMSTR configuration user interface 601 can include an option 618 to enable monitoring content consumption. In one or more embodiments, the content can include content from websites, such as media streaming websites, social media websites, podcasts, and so on. In one or more embodiments, the content can include content stored locally on the managed electronic device. In one or more embodiments, the consumed content is used as criteria in determining an emotional state. In one or more embodiments, the emotional state monitoring, tracking, and reporting configuration user interface 601 can include an option 620 to enable monitoring communication. In one or more embodiments, the communication can include incoming and/or outgoing text messages, voice conversations, video calls, and/or other types of communication. In one or more embodiments, audio information in voice conversations and/or video calls may be converted to text via a speech-to-text process. The converted text can then be analyzed via natural language processing (NLP), and/or other suitable techniques. In one or more embodiments, the monitored communication is used as criteria in determining an emotional state.

In one or more embodiments, the EMSTR configuration user interface 601 can include an option 622 to enable monitoring browser and search history. In one or more embodiments, the browser and search history can include a list of websites visited, search terms used in search engines, questions and prompts provided to machine learning systems such as ChatGPT, and so on. In one or more embodiments, browser and search history is used as criteria in determining an emotional state. In one or more embodiments, the EMSTR configuration user interface 601 can include an option 624 to enable monitoring the location of a managed user. In one or more embodiments, location is used as criteria in determining an emotional state. In one or more embodiments, the location data can be used in combination with biometric data. As an example, an elevated heart rate and breathing rate may be of concern if a user is at home. In contrast, if the user is at a gym, the elevated heart rate and breathing rate may be expected, and is thus not a cause to generate a vulnerability alert in that scenario.

The user interface 601 depicted in FIG. 6 shows various radio buttons for selection by a user. As an example, option 610 shows a selected option, while option 612 shows an unselected option. One or more embodiments may include other user interface elements instead of, or in addition to, the radio buttons shown in FIG. 6. In one or more embodiments, the user interface can include checkboxes which allow users to select multiple options independently. In one or more embodiments, the user interface can include text input fields that allow users to enter custom text or numbers. In one or more embodiments, the user interface can include number steppers that allow users to increase or decrease a numerical value by tapping on increment/decrement buttons. In some embodiments, the alert threshold field 604 may be implemented using number stepper user interface elements. In one or more embodiments, the user interface can include drop-down menus that present a list of options in a compact form. When clicked or tapped, a list appears from which users can select one option. In embodiments, the report interval 606 may be implemented using a drop-down menu. Other user interface elements are possible in disclosed embodiments.

In one or more embodiments, the managed electronic device is configured to: receive, from the managing device of the second user, a command for setting or adjusting one or more threshold values respectively associated with the one or more emotional states of the first user; and set or adjust the one or more threshold values associated with the one or more emotional states of the first user according to the received command. The one or more threshold values includes at least one of (i) one or more individual threshold values associated with respective one or more individual emotional states of the first user and (ii) an overall threshold value associated with an overall emotional state of the first user that is determined based on a combination of the one or more individual emotional states.

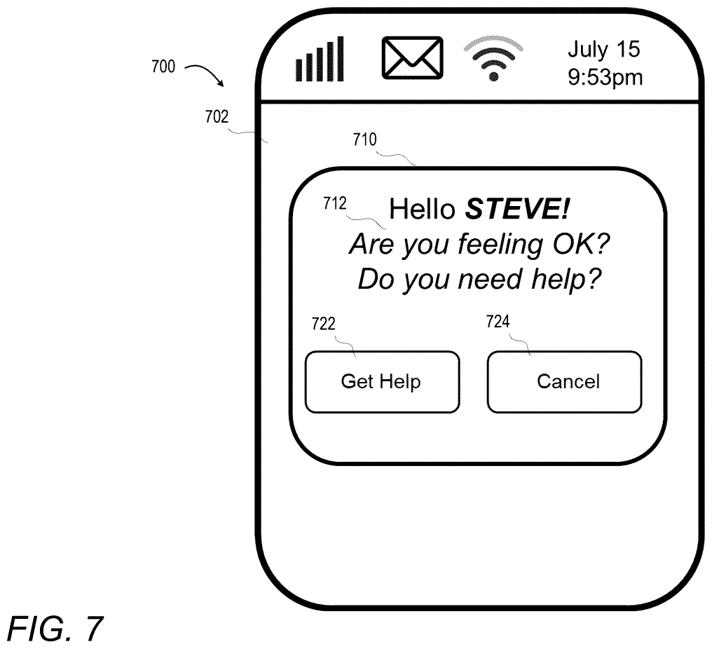

FIG. 7 is an example of an assistance query, according to one or more embodiments. An assistance query is a type of message that is sent directly to the managed device. In embodiments, the assistance query is used to reach a user who may be in a vulnerable state by rendering and presenting a message on his/her device. Device 700 may be a managed device. Device 700 may be similar to device 100 shown in FIG. 1. Device 700 includes an electronic display 702. Presented and rendered on display 702 is assistance query 710. In one or more embodiments, the assistance query 710 may be rendered and presented on the display 702 of device 700 when a vulnerable state is detected and option 612 of FIG. 6 is selected. The assistance query 710 may include a message 712 asking if the managed user needs assistance. In the example of FIG. 7, the message 712 says “Hello Steve! Are you feeling OK? Do you need help?” In one or more embodiments, an option is provided for the managing user to customize the message used in the assistance query 710. The assistance query 710 may further include an option 722 to get help. In one or more embodiments, the option 722, when invoked, may automatically call a previously configured telephone number, and/or send a message to a previously configured telephone number, email address, and/or other previously configured account. In one or more embodiments, the option 722, when invoked, may automatically call a suicide hotline (e.g., 988). The 988 telephone number is the nationwide, three-digit dialing code in the United States for the 988 Suicide and Crisis Lifeline. The 988 telephone number provides a direct connection to mental health support for anyone experiencing a mental health crisis, emotional distress, or suicidal thoughts. In this way, disclosed embodiments can provide fast intervention when an adverse emotional state is detected. The assistance query 710 may further include an option 724 to cancel the assistance query 710 and remove the assistance query 710 from the display 702.

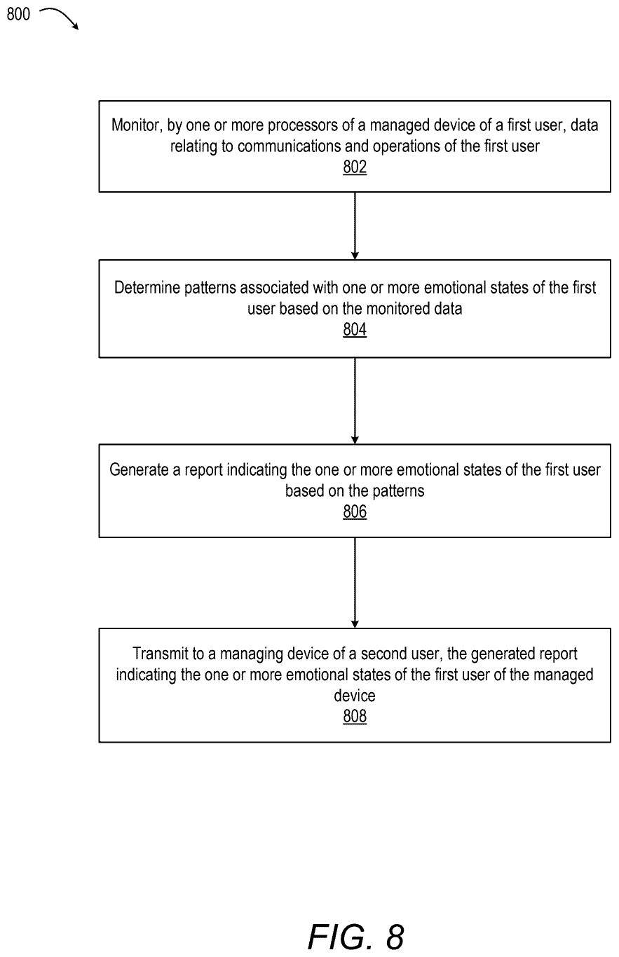

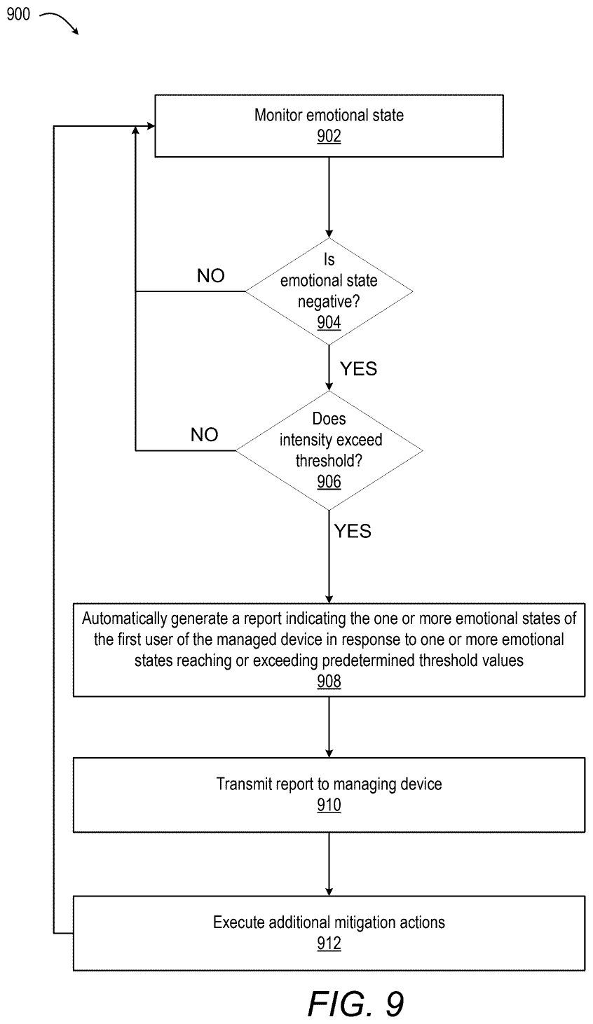

Referring now to the flowcharts presented by FIG. 8-FIG. 9, the descriptions of the methods in FIG. 8-FIG. 9 are provided with general reference to the specific components and features illustrated within the preceding FIG. 1-7. Specific components referenced in the methods of FIG. 8-FIG. 9 may be identical or similar to components of the same name used in describing preceding FIG. 1-7. In one or more embodiments, processor 102 (FIG. 1) configures electronic device 100 (FIG. 1) to provide the described functionality of the methods of FIG. 8-FIG. 9 by executing program code for one or more modules or applications provided within system memory 120 of electronic device 100, including emotional state monitoring, tracking, and reporting (ESMTR) module 152.

FIG. 8 depicts a flowchart of a computer-implemented method for monitoring, tracking, and reporting user emotional states from a managed device, according to one or more embodiments. The method 800 starts at block 802, where data relating to communications and/or operations of the first (managed) user is monitored and/or recorded. The data can include messaging/communications data pertaining to incoming and/or outgoing text messages, emoji usage, voice conversation data, video conversation data, and so on. The operations data can include content consumption data, browser activity data, search history data, machine learning prompt data, and so on. In one or more embodiments, the collected data comprises at least one of: at least one message communicated from or to the first user of the managed device, the at least one message being one from a group comprising incoming text, incoming voice, outgoing text, and outgoing voice communication; at least one browser search history of the first user of the managed device; at least one application activity of the first user of the managed device; at least one content consumed by the first user of the managed device; sensor data relating to the first user of the managed device; or a combination thereof.

The method 800 continues to block 804, where patterns associated with one or more emotional states of the first user are determined, based on the monitored data. The patterns can include determining a trend of a given emotional state over time. In one or more embodiments, the emotional state can include one of sadness, depression, anger, anxiety, fear, rage, and so on. In one or more embodiments, determining patterns associated with one or more emotional states of the first user of the managed device comprises: identifying, based on the monitored data, patterns triggering or leading to negative emotional states or distress; and determining whether the identified patterns triggering or leading to negative emotional states or distress reach or exceed one or more threshold values associated with the one or more emotional states of the first user.

The method 800 continues to block 806, where a report is generated that indicates the one or more emotional states of the first user, based on the patterns. In one or more embodiments, the report can include a list of detected emotions, a computed intensity level for each of the detected emotions, relevant incoming and/or outgoing messages that correspond to the detected emotions, consumed content that may correspond to the detected emotions, and/or other relevant information. Additionally, one or more embodiments may further include a recommendation for actions to take for improving the emotional state of the managed user. The method 800 continues to block 808, where the generated report is transmitted to a managing device, where the generated report indicates one or more emotional states of the first user, based on data relating to communications and operations of the first (managed) device.

FIG. 9 depicts a flowchart of a computer-implemented method for generating a report indicating emotional states based on collected data related to communications and operations on a managed user device, according to one or more embodiments. The method 900 starts a block 902, where the emotional state of the user associated with the managed device is performed via monitoring and analyzing data relating to communications and operations of the managed device. In one or more embodiments, monitoring data relating to communications and operations occurring on the managed device comprises collecting, via the one or more processors of the managed device, data relating to incoming and outgoing communications and user-initiated operations of the managed device, where the monitoring occurs based on a monitoring schedule established by the managing device. Also, in one or more embodiments, determining patterns associated with one or more emotional states of the first user comprises analyzing, via an artificial intelligence engine, the collected data to identify the one or more emotional states of the first user.

The method 900 continues to decision block 904, where a check is made to determine if the determined emotional state is negative. If, at decision block 904, the emotional state is deemed to not be negative, the method 900 returns to block 902 to continue monitoring the emotional state. If, at decision block 904, the emotional state is deemed to be negative, the method 900 continues to decision block 906, where a check is made to determine if the intensity value assigned to the emotional state exceeds a predetermined threshold. The check at decision block 906 can serve to prevent excessive ‘false positive’ vulnerable state alerts when the managed user is experiencing slightly negative emotions. If, at decision block 906, the emotional state is deemed not to exceed the predetermined threshold, the method 900 returns to block 902 to continue monitoring the emotional state. If, at decision block 906, the emotional state is deemed to exceed the predetermined threshold, the method 900 continues to block 908 where a report is generated indicating the emotional states of the managed user. In one or more embodiments, generating the report indicating the one or more emotional states of the first user of the managed device comprises: determining whether the one or more emotional states of the first user have reached or exceeded one or more threshold values corresponding to one or more emotional states; and automatically generating the report in response to at least one of the one or more emotional states reaching or exceeding a respective at least one of the one or more threshold values.

The method 900 then continues to block 910 where the report is transmitted to the managing device, thereby allowing the managing person (e.g., parent, caregiver, etc.) to be notified of the emotional state of the managed user. The method 900 then continues to block 912, where one or more additional mitigation actions can be executed. The additional mitigation actions can include, but are not limited to, blocking access to a website, blocking access to a content channel, blocking access to communication to/from another person/device/account, blocking access to an application, sending an assistance query to the managed device, and/or other suitable mitigation actions. The method 900 then returns to block 902 to continue monitoring the emotional state of the managed user. In one or more embodiments, the monitoring may be continuous. In one or more embodiments, options are provided for the managing user to specify a desired duration and/or monitoring schedule for performing the emotional state monitoring of the managed user.

As can now be appreciated, disclosed embodiments provide techniques for monitoring, tracking, and reporting user emotional states from a managed device. Embodiments monitor communication and/or operation activities and alert another person such as a caregiver or parent, in response to detecting a negative emotional state that exceeds a predetermined level. Embodiments can further provide recommendations, and/or perform other mitigation actions such as blocking callers associated with harmful and/or toxic communication patterns, blocking consumption of content associated with harmful emotions, and rendering and presenting an assistance query to the managed user. Early intervention is critical in preventing a person from becoming depressed or despondent due to bullying, social media consumption, exposure to certain types of online content, and/or harmful communication like cyberbullying. As children and teenagers spend increasing amounts of time online, they become more vulnerable to negative influences that can impact their mental health and well-being. Disclosed embodiments help provide this early intervention, enabling caregivers/parents the opportunity to provide guidance, support, and education to help their children navigate the digital world safely, promote mental health and well-being, and reduce the risk of the children causing harm to themselves or others.

In the above-described methods, one or more of the method processes may be embodied in a computer readable device containing computer readable code such that operations are performed when the computer readable code is executed on a computing device. In some implementations, certain operations of the methods may be combined, performed simultaneously, in a different order, or omitted, without deviating from the scope of the disclosure. Further, additional operations may be performed, including operations described in other methods. Thus, while the method operations are described and illustrated in a particular sequence, use of a specific sequence or operations is not meant to imply any limitations on the disclosure. Changes may be made with regards to the sequence of operations without departing from the spirit or scope of the present disclosure. Use of a particular sequence is therefore, not to be taken in a limiting sense, and the scope of the present disclosure is defined primarily by the appended claims.

Aspects of the present disclosure are described above with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. Computer program code for carrying out operations for aspects of the present disclosure may be written in any combination of one or more programming languages, including an object-oriented programming language, without limitation. These computer program instructions may be provided to a processor of a general-purpose computer, special-purpose computer, or other programmable data processing apparatus to produce a machine that performs the method for implementing the functions/acts specified in the flowchart and/or block diagram block or blocks. The methods are implemented when the instructions are executed via the processor of the computer or other programmable data processing apparatus.

As will be further appreciated, the processes in embodiments of the present disclosure may be implemented using any combination of software, firmware, or hardware. Accordingly, aspects of the present disclosure may take the form of an entirely hardware embodiment or an embodiment combining software (including firmware, resident software, micro-code, etc.) and hardware aspects that may all generally be referred to herein as a “circuit,” “module,” or “system.” Furthermore, aspects of the present disclosure may take the form of a computer program product embodied in one or more computer readable storage device(s) having computer readable program code embodied thereon. Any combination of one or more computer readable storage device(s) may be utilized. The computer readable storage device may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage device can include the following: a portable computer diskette, a hard disk, a random-access memory (RAM), a read-only memory (ROM), an erasable programmable read-only memory (EPROM or Flash memory), a portable compact disc read-only memory (CD-ROM), an optical storage device, a magnetic storage device, or any suitable combination of the foregoing. In the context of this document, a computer readable storage device may be any tangible medium that can contain or store a program for use by or in connection with an instruction execution system, apparatus, or device.

Where utilized herein, the terms “tangible” and “non-transitory” are intended to describe a computer-readable storage medium (or “memory”) excluding propagating electromagnetic signals, but are not intended to otherwise limit the type of physical computer-readable storage device that is encompassed by the phrase “computer-readable medium” or memory. For instance, the terms “non-transitory computer readable medium” or “tangible memory” are intended to encompass types of storage devices that do not necessarily store information permanently, including, for example, RAM. Program instructions and data stored on a tangible computer-accessible storage medium in non-transitory form may afterwards be transmitted by transmission media or signals such as electrical, electromagnetic, or digital signals, which may be conveyed via a communication medium such as a network and/or a wireless link.

The description of the present disclosure has been presented for purposes of illustration and description, but is not intended to be exhaustive or limited to the disclosure in the form disclosed. Many modifications and variations will be apparent to those of ordinary skill in the art without departing from the scope of the disclosure. The described embodiments were chosen and described in order to best explain the principles of the disclosure and the practical application, and to enable others of ordinary skill in the art to understand the disclosure for various embodiments with various modifications as are suited to the particular use contemplated.

As used herein, the term “or” is inclusive unless otherwise explicitly noted. Thus, the phrase “at least one of A, B, or C” is satisfied by any element from the set {A, B, C} or any combination thereof, including multiples of any element.

While the disclosure has been described with reference to example embodiments, it will be understood by those skilled in the art that various changes may be made and equivalents may be substituted for elements thereof without departing from the scope of the disclosure. In addition, many modifications may be made to adapt a particular system, device, or component thereof to the teachings of the disclosure without departing from the scope thereof. Therefore, it is intended that the disclosure not be limited to the particular embodiments disclosed for carrying out this disclosure, but that the disclosure will include all embodiments falling within the scope of the appended claims.

What is claimed is:

1. An electronic device comprising:
a communications subsystem enabling the electronic device to communicatively connect to at least one second electronic device, wherein the electronic device is a managed electronic device and wherein the at least one second electronic device is a managing electronic device;
a memory having stored thereon an emotional state monitoring, tracking, and reporting (ESMTR) module that autonomously records data associated with an emotional state of a first user of the electronic device;
at least one processor coupled to the communications subsystem and the memory and which processes program code of the ESMTR module, the at least one processor configured to cause the electronic device to:
monitor and record data relating to communications and operations occurring on the electronic device of the first user;
determine patterns associated with one or more emotional states of the first user based on the monitored data;
generate a report indicating the one or more emotional states of the first user based on the determined patterns; and
transmit, to a managing device of a second user, the generated report indicating the one or more emotional states of the first user of the managed electronic device.

2. The electronic device of claim 1, wherein to monitor and record data relating to communications occurring on the electronic device of the first user, the at least one processor is further configured to obtain data from at least one outgoing message.

3. The electronic device of claim 2, wherein to determine patterns associated with one or more emotional states of the first user based on the monitored data, the at least one processor is further configured to:
perform a sentiment analysis on the at least one outgoing message; and
determine a sentiment score based on the sentiment analysis.

4. The electronic device of claim 3, wherein the at least one processor is further configured to:
in response to the sentiment score being above a predetermined threshold:
generate a vulnerable state alert message; and
transmit the vulnerable state alert message to the at least one second electronic device.

5. The electronic device of claim 3, wherein to determine the patterns associated with the one or more emotional states of the first user of the managed device, the at least one processor is further configured to:
receive, from the managing device of the second user, a command for setting or adjusting one or more threshold values respectively associated with the one or more emotional states of the first user; and
set or adjust the one or more threshold values associated with the one or more emotional states of the first user according to the received command;
wherein the one or more threshold values comprise at least one of (i) one or more individual threshold values associated with respective one or more individual emotional states of the first user and (ii) an overall threshold value associated with an overall emotional state of the first user that is determined based on a combination of the one or more individual emotional states.

6. The electronic device of claim 2, wherein to determine patterns associated with one or more emotional states of the first user based on the monitored data, the at least one processor is further configured to:
obtain data from at least one incoming message;
perform a sentiment analysis on the at least one incoming message; and
determine a sentiment score based on the sentiment analysis of the at least one outgoing message and the at least one incoming message.

7. The electronic device of claim 2, wherein to determine patterns associated with one or more emotional states of the first user based on the monitored data, the at least one processor is further configured to monitor biometric data from among a group comprising at least one of heart rate, electrodermal activity, and respiratory pattern.

8. The electronic device of claim 1, wherein to monitor and record data relating to operations occurring on the electronic device of the first user, the at least one processor is further configured to monitor at least one of:
at least one browser search history of the first user of the electronic device;
at least one application activity of the first user of the electronic device; and
at least one content consumed by the first user of the electronic device.

9. The electronic device of claim 1, wherein the at least one processor is further configured to:
generate, via an artificial intelligence engine, a recommendation indicating one or more remedial actions that can be taken by the second user of the managing device to address the one or more emotional states of the first user of the managed device; and
transmit the recommendation to the managing device along with the report.

10. The electronic device of claim 1, wherein to generate a report indicating the one or more emotional states of the first user of the managed device, the at least one processor is further configured to provide within the report at least one of:
a description of the one or more emotional states of the first user;
a timeframe for which the one or more emotional states of the first user has been ongoing;
a root cause of, or suspected trigger for the one or more emotional states of the first user; and
the monitored data that caused or triggered the one or more emotional states of the first user.

11. A method comprising:
monitoring, by one or more processors of a managed device of a first user, data relating to communications and operations occurring on the managed device of the first user;
determining patterns associated with one or more emotional states of the first user based on the monitored data;
generating a report indicating the one or more emotional states of the first user based on the patterns; and
transmitting, to a managing device of a second user, the generated report indicating the one or more emotional states of the first user of the managed device.

12. The method of claim 11, wherein:
monitoring data relating to communications and operations occurring on the managed device comprises collecting, via the one or more processors of the managed device, data relating to incoming and outgoing communications and user-initiated operations of the managed device wherein the monitoring occurs based on a monitoring schedule established by the managing device; and
determining patterns associated with one or more emotional states of the first user comprises analyzing, via an artificial intelligence engine, the collected data to identify the one or more emotional states of the first user.

13. The method of claim 12, wherein the collected data comprises at least one of:
at least one message communicated from or to the first user of the managed device, the at least one message being one from a group comprising incoming text, incoming voice, outgoing text, and outgoing voice communication;
at least one browser search history of the first user of the managed device;
at least one application activity of the first user of the managed device;
at least one content consumed by the first user of the managed device;
sensor data relating to the first user of the managed device; or
a combination thereof.

14. The method of claim 12, wherein analyzing the collected data comprises:
performing a sentiment analysis based on the collected data; and
determining a dominant emotion of the first user based on the sentiment analysis.

15. The method of claim 11, wherein determining patterns associated with one or more emotional states of the first user of the managed device comprises:
identifying, based on the monitored data, patterns triggering or leading to negative emotional states or distress; and
determining whether the identified patterns triggering or leading to negative emotional states or distress reach or exceed one or more threshold values associated with the one or more emotional states of the first user.

16. The method of claim 15, wherein determining the patterns associated with the one or more emotional states of the first user of the managed device comprises:
receiving, from the managing device of the second user, a command for setting or adjusting the one or more threshold values respectively associated with the one or more emotional states of the first user; and
setting or adjusting the one or more threshold values associated with the one or more emotional states of the first user according to the received command;
wherein the one or more threshold values comprise at least one of (i) one or more individual threshold values associated with respective one or more individual emotional states of the first user and (ii) an overall threshold value associated with an overall emotional state of the first user that is determined based on a combination of the one or more individual emotional states.

17. The method of claim 11, wherein generating a report indicating the one or more emotional states of the first user of the managed device comprises incorporating into the report at least one of:
a description of the one or more emotional states of the first user;
a timeframe for which the one or more emotional states of the first user has been ongoing;
a root cause of, or suspected trigger for the one or more emotional states of the first user; and
the monitored data that caused or triggered the one or more emotional states of the first user.

18. The method of claim 17, wherein generating the report indicating the one or more emotional states of the first user of the managed device further comprises:
generating, via an artificial intelligence engine, a recommendation indicating one or more remedial actions that can be taken by the second user of the managing device to address the one or more emotional states of the first user of the managed device.

19. The method of claim 11, wherein generating the report indicating the one or more emotional states of the first user of the managed device comprises:
determining whether the one or more emotional states of the first user have reached or exceeded one or more threshold values corresponding to one or more emotional states; and
automatically generating the report in response to at least one of the one or more emotional states reaching or exceeding a respective at least one of the one or more threshold values.

20. A computer program product comprising:
a non-transitory computer readable medium; and
program code on the computer readable medium that when processed by a processor of a communication device configures the processor to perform functions of:
monitoring, by one or more processors of a managed device of a first user, data relating to communications and operations occurring on the managed device of the first user;
determining patterns associated with one or more emotional states of the first user based on the monitored data;
generating a report indicating the one or more emotional states of the first user based on the patterns; and
transmitting, to a managing device of a second user, the generated report indicating the one or more emotional states of the first user of the managed device.
